(19) United States
(12) Patent Application Publication
(10) Pub. No.: US 2025/0228881 A1
(43) Pub. Date: Jul. 17, 2025

(54) COMPOSITION FOR PREVENTION OR TREATMENT OF MULTIPLE MYELOMA COMPRISING NOVEL TETRACYCLIC TRITERPENE COMPOUND AS ACTIVE INGREDIENT

(71) Applicants: KBLUEBIO INC, Jeollanam-do (KR); INDUSTRY FOUNDATION OF CHONNAM NATIONAL UNIVERSITY, Gwangju (KR)

(72) Inventors: Hye Ran KIM, Gwangju (KR); Young Eun LEE, Gwangju (KR); Myung Geun SHIN, Gwangju (KR)

(21) Appl. No.: 18/710,538

(22) PCT Filed: Nov. 17, 2022
(86) PCT No.: PCT/KR2022/018180

§ 371 Date: May 15, 2024

(30) Foreign Application Priority Data
Nov. 19, 2021 (KR) 1020210160623

(51) Int. Cl. A61K 31/704; A61K 31/4439; A61P 35/00

(52) U.S. Cl. CPC A61K 31/704; A61K 31/4439; A61P 35/00

(57) ABSTRACT

A novel multi-fused-ring compound and a composition for preventing or treating multiple myeloma comprising the same as an active ingredient is described herein. The compound is a derivative synthesized from the natural compound ginsenoside as a starting material, and shows no cytotoxicity to normal hematopoietic stem cells, while exhibiting a significant killing effect on various multiple myeloma cell lines, indicating that it may be administered for a long period of time without side effects. In addition, the compound exhibits a significant synergistic effect when co-administered with the immunomodulator thalidomide or its analog lenalidomide, and thus may be useful as an efficient therapeutic agent or therapeutic aid agent composition for multiple myeloma, an incurable disease.

### CROSS REFERENCE TO RELATED APPLICATIONS

The present application is a national stage entry according to 35 U.S.C. § 371 of PCT application No.: PCT/KR2022/018180 filed on Nov. 17, 2022; which claims priority to Korean Patent Application Serial No.: 10-2021-0160623, which was filed on Nov. 19, 2021; which are incorporated herein by reference in their entirety and for all purposes.

### TECHNICAL FIELD

The present invention relates to a novel compound having a four-fused-ring structure and a composition comprising the same for preventing or treating bone marrow cancer, including multiple myeloma.

### BACKGROUND

Among blood cancers that occur in adults, the incidence of multiple myeloma has increased most rapidly with population aging. Treatment of multiple myeloma has been attempted based on melphalan and prednisone therapy, but anticancer drugs based on the immunomodulators thalidomide and lenalidomide and the proteasome inhibitor bortezomib are widely being used in clinical practice. However, these drugs cause pancytopenia by destroying normal hematopoietic stem cells or inhibiting their function, and also cause serious side effects such as the occurrence of peripheral neuritis, embolism/thrombosis, and secondary primary cancer. In addition, as the drug resistance of multiple myeloma increases, mortality due to multiple myeloma is also significantly increasing.

Accordingly, in order to successfully treat multiple myeloma and to improve patient survival rates, the development of new anticancer drugs with dramatically reduced side effects mentioned above is required. Therefore, the present invention is intended to provide an efficient therapeutic composition for multiple myeloma, which is an incurable blood cancer, by discovering a novel small-molecule compound that exhibits a significant killing effect on multiple myeloma cells, verifying the anticancer effect of the compound in various ways through cell line and animal experiments, and observing that the compound shows no significant toxicity to a group of peripheral blood hematopoietic stem cells derived from a normal donor.

Throughout the present specification, a number of publications and patent documents are referred to and cited. The disclosure of the cited publications and patent documents is incorporated herein by reference in its entirety to more clearly describe the state of the art to which the present invention pertains and the content of the present invention.

### SUMMARY

The present inventors have made extensive research efforts to develop an effective and side-effect-free small-molecule compound for the treatment of incurable blood cancers caused by abnormal proliferation of plasma cells, including multiple myeloma. As a result, the present inventors have found that a tetracyclic triterpene compound represented by Formula (1) below exhibits a significant killing effect on various multiple myeloma cell lines without showing significant toxicity to normal hematopoietic stem cells, and exhibits a therapeutic effect better than a commercially available drug in vivo, thereby completing the present invention.

Therefore, an object of the present invention is to provide a novel tetracyclic triterpene compound represented by Formula (1) below and a composition for preventing or treating multiple myeloma comprising the same as an active ingredient.

Other objects and advantages of the present invention will be more apparent from the following detailed description, the appended claims, and the accompanying drawings.

### DETAILED DESCRIPTION

According to one aspect of the present invention, the present invention provides a composition for preventing or treating cancer comprising a compound represented by the following Formula 1 or a pharmaceutically acceptable salt thereof as an active ingredient:

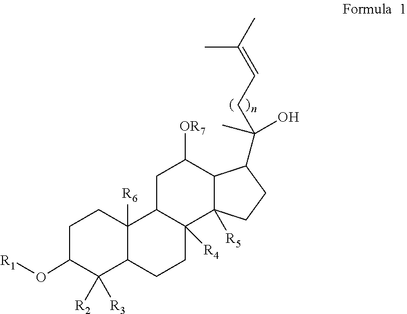

In the Formula, R₁ is

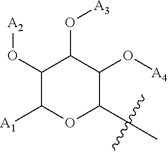

(where A₁ is C₁-C₃ alkyl unsubstituted or substituted with hydroxy, and A₂ to A₄ are each independently hydrogen or C₁-C₃ alkyl); R₂ to R₇ are each independently hydrogen or C₁-C₃ alkyl; and n is an integer ranging from 1 to 3.

The present inventors have made extensive research efforts to develop an effective and side-effect-free small-molecule compound for the treatment of incurable blood cancers caused by abnormal proliferation of plasma cells, including multiple myeloma. As a result, the present inventors have found that the compound represented by Formula (1) exhibits a significant killing effect on various multiple myeloma cell lines without showing significant toxicity to normal hematopoietic stem cells, and exhibits a therapeutic effect better than a commercially available drug in vivo.

In the present specification, the term “alkyl” refers to a straight-chain or branched saturated hydrocarbon group, and includes, for example, methyl, ethyl, propyl, isopropyl, etc. The term “C₁-C₃ alkyl” refers to an alkyl group having an alkyl unit having 1 to 3 carbon atoms, and when the C₁-C₃ alkyl is substituted, the carbon atom number of the substituent is not included.

According to a specific embodiment of the present invention, A₁ is hydroxymethyl, and A₂ to A₄ are each hydrogen.

According to a specific embodiment of the present invention, R₂ to R₆ are each independently hydrogen or methyl.

According to a specific embodiment of the present invention, n is 2.

More specifically, the compound represented by Formula 1 is a compound represented by the following Formula 2:

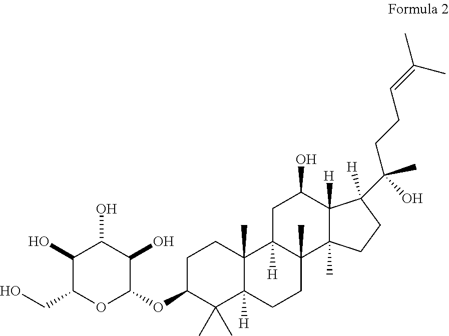

In the present specification, the term “pharmaceutically acceptable salt” includes a salt derived from a pharmaceutically acceptable inorganic acid, organic acid, or base. Examples of suitable acids include hydrochloric acid, hydrobromic acid, sulfuric acid, nitric acid, perchloric acid, fumaric acid, maleic acid, phosphoric acid, glycolic acid, lactic acid, salicylic acid, succinic acid, toluene-p-sulfonic acid, tartaric acid, acetic acid, trifluroacetic acid, citric acid, methanesulfonic acid, formic acid, benzoic acid, malonic acid, naphthalene-2-sulfonic acid, benzenesulfonic acid, and the like. Salts derived from suitable bases include salts of alkali metals such as sodium, alkaline earth metals such as magnesium, ammonium, and the like.

In the present specification, the term “prevention” means inhibiting the occurrence of a disorder or a disease in a subject who has never been diagnosed as having the disorder or disease, but is likely to suffer from such disorder or disease.

In the present specification, the term “treatment” means (a) inhibiting the progress of a disorder, disease or symptom; (b) alleviating the disorder, disease or symptom; or (c) eliminating the disorder, disease or symptom. The composition of the present invention functions to inhibit the proliferation of tumor cell lines, induce apoptosis, and reduce the tumor lesion area, thereby inhibiting the progress of symptoms caused by tumors, or eliminating or alleviating the symptoms. Thus, the composition of the present invention may serve as a therapeutic composition for the disease alone, or may be administered in combination with other pharmacological ingredients having anticancer effects and applied as a therapeutic aid for the disease. Accordingly, as used in the present specification, the term “treatment” or “therapeutic agent” encompasses the meaning of “treatment aid” or “therapeutic aid agent”.

In the present specification, the term “administration” or “administering” means administering a therapeutically effective amount of the composition of the present invention directly to a subject so that the same amount is formed in the subject's body.

In the present specification, the term “therapeutically effective amount” refers to an amount of the composition sufficient to provide a therapeutic or prophylactic effect to a subject to whom/which the composition of the present invention is to be administered. Accordingly, the term “therapeutically effective amount” is meant to include a “prophylactically effective amount”.

In the present specification, the term “subject” includes, without limitation, humans, mice, rats, guinea pigs, dogs, cats, horses, cows, pigs, monkeys, chimpanzees, baboons or rhesus monkeys. Specifically, the subject of the present invention is a human.

According to a specific embodiment of the present invention, the cancer to be prevented or treated by the composition of the present invention is blood cancer.

In the present specification, the term “blood cancer” refers to a malignant tumor that occurs in white blood cells, red blood cells, and platelets, which are components of blood; bone marrow where blood is produced; or the lymphatic system including lymphocytes, lymph nodes, and lymphatic vessels, which make up the immune system.

More specifically, the blood cancer to be prevented or treated by the composition of the present invention is bone marrow cancer. Most specifically, the blood cancer is multiple myeloma.

In the present specification, the term “multiple myeloma” refers to blood cancer that is caused by abnormal differentiation and proliferation of plasma cells, which is a type of white blood cell responsible for the immune system, in the bone marrow.

According to a specific embodiment of the present invention, the composition of the present invention further comprises a compound represented by the following Formula 3 or a pharmaceutically acceptable salt thereof:

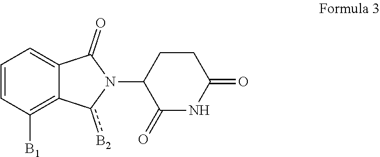

In the Formula, B₁ is hydrogen or —NH₂, [symbol] represents a single bond or double bond, and B₂ is hydrogen when [symbol] represents a single bond, or oxygen when [symbol] represents a double bond.

More specifically, B₁ is —NH₂, and B₂ is hydrogen. According to the octet rule, it is obvious that when B₂ is hydrogen, [symbol] represents a single bond. The compound of Formula 3, wherein B₁ is —NH₂ and B₂ is hydrogen, is lenalidomide (C₁₃H₁₃N₃O₃; 3-(4-amino-1-oxoisoindolin-2-yl) piperidine-2,6-dione).

Lenalidomide is an immunomodulator widely used in the treatment of multiple myeloma and myelodysplastic syndrome, and has anti-angiogenic and anti-inflammatory activities. According to the present invention, the compound of the present invention not only exhibited a therapeutic effect equal to or higher than that of lenalidomide, a representative commercially available therapeutic agent for multiple myeloma, but also exhibited a significant synergistic effect when co-administered with lenalidomide. Accordingly, the compound of the present invention may not only be applied as an anticancer agent alone, but may also be useful as an efficient therapeutic aid agent that is co-administered with lenalidomide or similar immunomodulators.

According to another specific embodiment of the present invention, B₁ is hydrogen and B₂ is oxygen. According to the octet rule, it is obvious that when B₂ is oxygen, [symbol] represents a double bond. The compound of Formula 3, wherein B₁ is hydrogen and B₂ is oxygen, is thalidomide (C₁₃H₁₀N₂O₄; 2-(2,6-dioxopiperidin-3-yl)-1H-isoindole-1,3(2H)-dione).

Thalidomide is an analogue, from which lenalidomide is derived, and has been used for a long time in the treatment of multiple myeloma until lenalidomide was approved in the United States in 2005.

When the composition of the present invention is prepared as a pharmaceutical composition, the pharmaceutical composition of the present invention comprises a pharmaceutically acceptable carrier. Examples of the pharmaceutically acceptable carrier that is comprised in the pharmaceutical composition of the present invention include, but are not limited to, lactose, dextrose, sucrose, sorbitol, mannitol, starch, gum acacia, calcium phosphate, alginate, gelatin, calcium silicate, microcrystalline cellulose, polyvinylpyrrolidone, cellulose, water, syrup, methyl cellulose, methylhydroxybenzoate, propylhydroxybenzoate, talc, magnesium stearate, and mineral oil, which are commonly used in formulation. The pharmaceutical composition of the present invention may further comprise a lubricant, a wetting agent, a sweetening agent, a flavoring agent, an emulsifying agent, a suspending agent, a preservative, and the like, in addition to the above-described ingredients. Suitable pharmaceutically acceptable carriers and agents are described in detail in Remington's Pharmaceutical Sciences (19^{th} ed., 1995).

The pharmaceutical composition of the present invention may be administered via various routes. Specifically, it may be administered parenterally. More specifically, it may be administered orally, intravenously, intra-arterially, subcutaneously, intraperitoneally, intradermally, intramuscularly, intracerebroventricularly, intrathecally, by inhalation, intranasally, intra-articularly, or topically.

An appropriate dosage of the pharmaceutical composition of the present invention may vary depending on various factors such as formulation method, administration mode, patient's age, weight, sex, pathological condition, diet, administration time, administration route, excretion rate, and reaction sensitivity. A preferred dosage of the pharmaceutical composition of the present invention is within the range of 0.0001 to 100 mg/kg for an adult.

The pharmaceutical composition of the present invention may be prepared in a unit dose form or prepared to be contained in a multi-dose container by formulating with a pharmaceutically acceptable carrier and/or excipient, according to a method that may be easily carried out by a person skilled in the art. In this case, the formulation of the pharmaceutical composition may be a solution, suspension, syrup or emulsion in oil or aqueous medium, or an extract, powder, granule, tablet or capsule, and may further comprise a dispersing agent or a stabilizer.

According to another aspect of the present invention, the present invention provides an anticancer composition for co-administration with a compound represented by the following Formula 3 or a pharmaceutically acceptable salt thereof, comprising the above-described compound of Formula 1 or pharmaceutically acceptable salt thereof according to the present invention as an active ingredient:

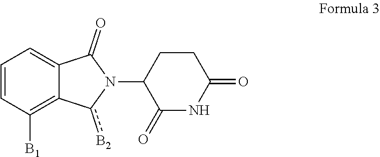

The compounds of Formula 1 and Formula 3 used in the present invention and the type of cancer that can be prevented or treated by these compounds have already been described in detail above, and thus the description thereof will be omitted to avoid excessive overlapping.

As described above, the compound of the present invention exhibited a significant synergistic effect when co-administered with lenalidomide. Thus, the compound of the present invention may be co-administered with phthalimide-based compounds, including lenalidomide, in order to maximize the anticancer effect and improve patient survival rates.

Co-administration may be performed by administering a single formulation comprising both the compound of Formula 1 and compound of Formula 3 according to the present invention, or may be performed by administering separate formulations comprising each individual compound simultaneously or sequentially in any order with an appropriate time gap.

According to another aspect of the present invention, the present invention provides a method for preventing or treating cancer, comprising a step of administering to a subject a compound represented by the following Formula 1 or a pharmaceutically acceptable salt thereof:

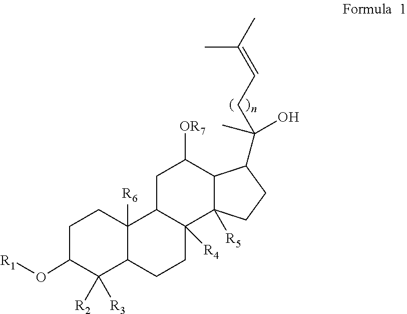

The compound of Formula 1 used in the present invention and the type of cancer that can be prevented or treated by the compound have already been described in detail above, and thus the description thereof will be omitted to avoid excessive overlapping.

According to a specific embodiment of the present invention, the method of the present invention further comprises a step of administering to the subject a compound represented by the following Formula 3 or a pharmaceutically acceptable salt thereof:

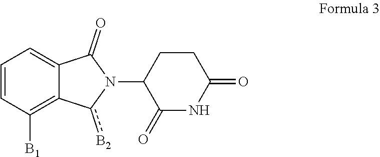

The compound of Formula 3 used in the present invention has already been described in detail above, and thus the description thereof will be omitted to avoid excessive overlapping.

### Advantageous Effects

The features and advantages of the present invention are summarized as follows:
(a) The present invention provides a novel tetracyclic triterpene compound and a composition for preventing or treating multiple myeloma comprising the same as an active ingredient.
(b) The compound of the present invention is a triterpene derivative synthesized from the natural compound ginsenoside as a starting material, and shows no cytotoxicity to normal hematopoietic stem cells while exhibiting a significant killing effect on various multiple myeloma cell lines, indicating that it may be administered for a long period of time without side effects.
(c) In addition, the compound of the present invention exhibits a significant synergistic effect when co-administered with the immunomodulator thalidomide or its analog lenalidomide, and thus may be useful as an efficient therapeutic agent or therapeutic aid agent composition for multiple myeloma, an incurable disease.

### BRIEF DESCRIPTION OF THE DRAWINGS

FIG. 1 shows spectroscopic data of KBB-N2, a small-molecule compound discovered in the present invention.

FIG. 2 shows the results of analyzing the chemical characteristics of KBB-N2.

FIG. 3 shows the results of measuring the killing effect of KBB-N2 at each concentration against multiple myeloma cell lines RPMI 8226 (FIG. 3a) and IM9 (FIG. 3b).

FIG. 4 shows the results of measuring the killing effects of KBB-N2-1, KBB-N2-4, KBB-N2-5 and KBB-N2-6, which are KBB-N2 derivative compounds, against U266 (FIGS. 4a and 4c), RPMI 8226 (FIGS. 4b and 4d) and IM9 (FIG. 4e), which are multiple myeloma cell lines.

FIG. 5 shows the results of evaluating the toxicity of KBB-N2 to normal hematopoietic stem cells. Specifically, FIG. 5 shows the results of confirming that KBB-N2 is not toxic to normal hematopoietic stem cells (FIG. 5a), and shows cell viability as a function of drug concentration (FIG. 5b).

FIG. 6 shows the results of evaluating the effect of co-administration of KBB-N2 and lenalidomide. Specifically, FIG. 6 shows the death rate of multiple myeloma cells by administration of lenalidomide alone (FIF. 6a), the death rate of multiple myeloma cells by each combination of KBB-N2 and lenalidomide (FIGS. 6b and 6c), and apoptosis by each combination (FIGS. 6d and 6e).

FIG. 7 is a schematic diagram showing an animal experiment process applied in the present invention.

FIG. 8 shows the results of administering KBB-N2 to the tail vein of multiple myeloma mouse models established by injecting the tail vein of combined immunodeficient mice with an RPMI 8226 cell line (#Cat.SC060-LG) (GenTarget Inc.; San Diego, CA, USA) into which luciferase gene, green fluorescent protein (GFP), and puromycin resistance gene have been inserted. Specifically, FIG. 8 shows bioluminescence imaging results for a control group, a KBB-N2-administered group, and a lenalidomide-administered group (FIG. 8a), changes in bioluminescence intensity, body weight, and survival rate (FIG. 8b), and the results of examining bone marrow tissue in a KBB-N2-adminstered multiple myeloma mouse model by immunohistochemical staining (FIG. 8c)

FIG. 9 shows a heat map (FIG. 9a) and a gene list (FIG. 9b) for genes expressed differentially between a 50 μM KBB-N2-administered group and a non-KBB-N2-administered group.

FIG. 10 is a schematic diagram showing the KBB-N2-induced death mechanism of multiple myeloma cells.

FIG. 11 shows the death rate of multiple myeloma cells by administration of KBB-N2 alone or cisplatin alone.

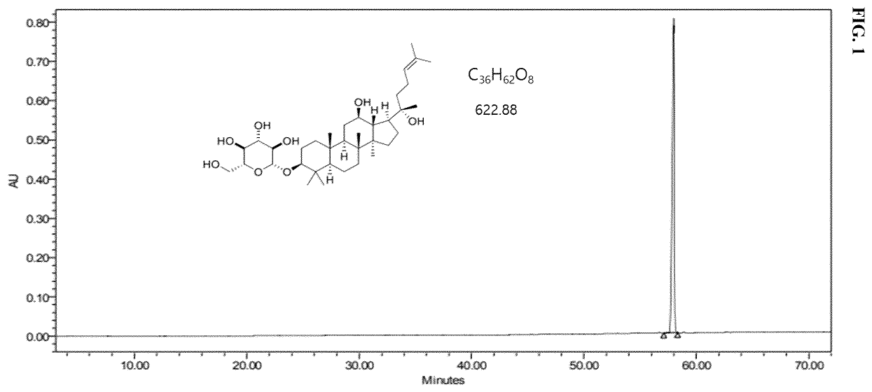

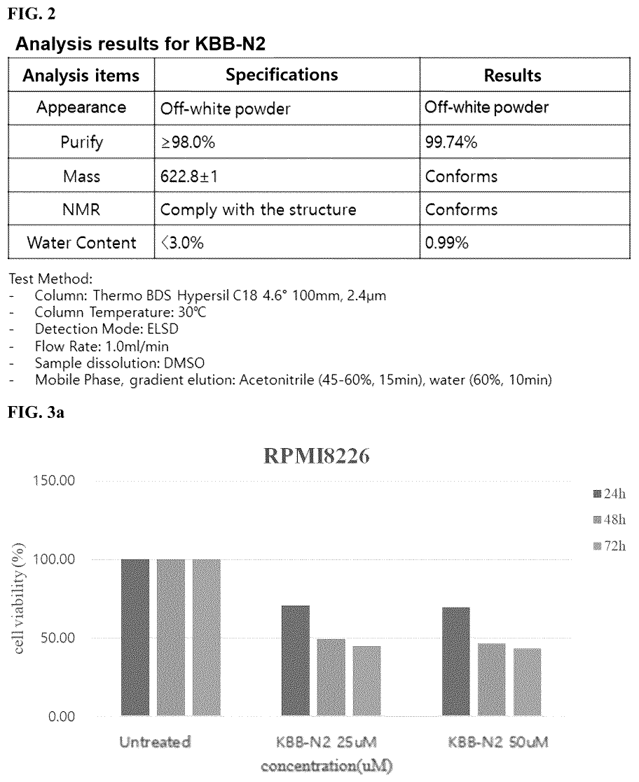

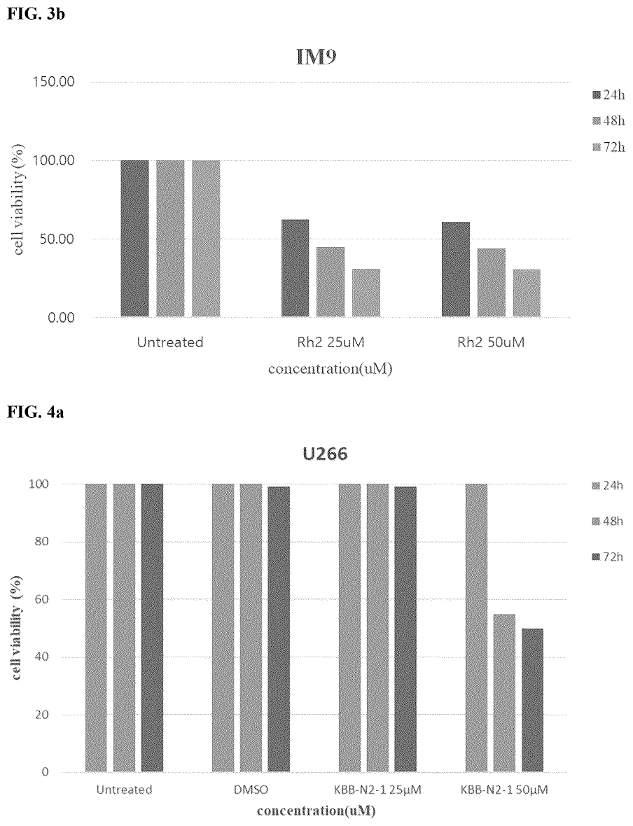

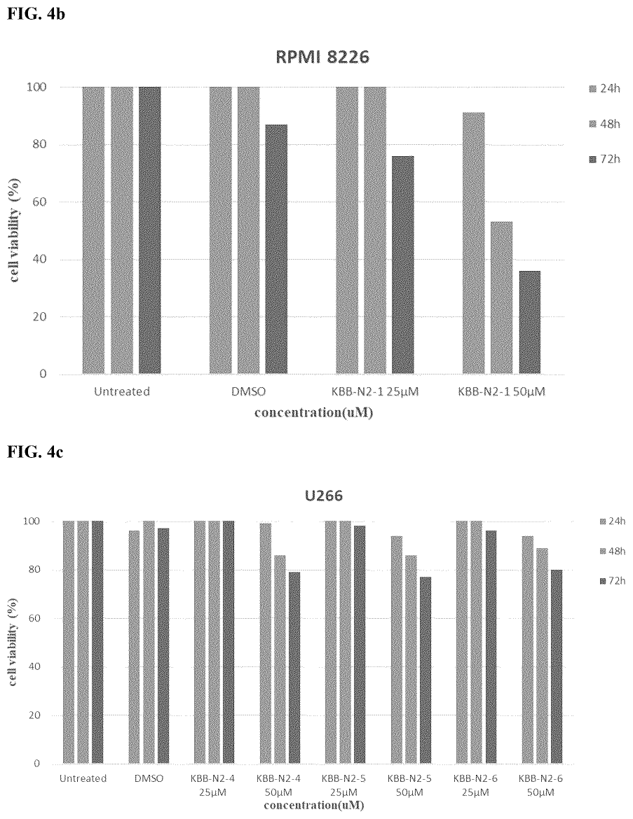

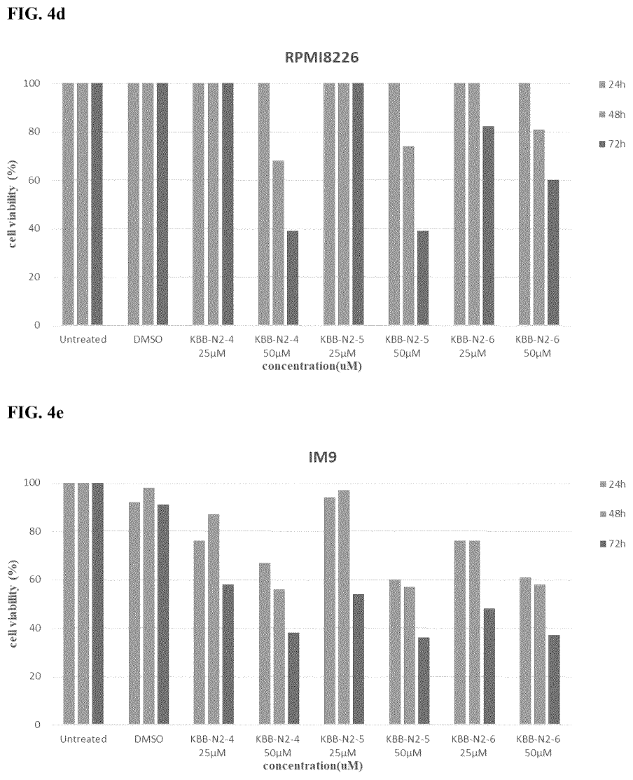

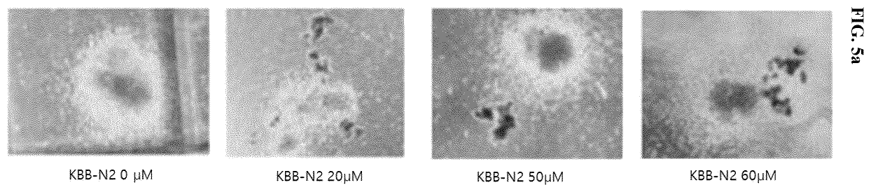

### MODE FOR INVENTION

Hereinafter, the present invention will be described in more detail by way of examples. These examples are only for explaining the present invention in more detail, and it will be apparent to those skilled in the art that the scope of the present invention according to the subject matter of the present invention is not limited by these examples.

### Examples

### Synthesis of KBB-N2 Compound

60% NaH (15 mg, 0.65 mmol) and BnBr (0.03 mL, 0.26 mmol) were added to a solution of PPD (100 mg, 0.22 mmol) in N,N-dimethylformamide at 0° C. Then, the mixture was stirred at room temperature for 2 hours. The reaction was terminated by adding a saturated aqueous solution of ammonium chloride, and the resulting mixture was extracted three times with EA. The obtained organic layer was washed with water and brine and dried over sodium sulfate. The solvent was evaporated under vacuum, and the residue was purified by silica gel column chromatography using ethyl acetate/hexane to obtain a pure compound (112 mg, 93.7%).

### Cell Lines and Cell Culture

Human multiple myeloma cell lines U266 (ATCC CCL-155), IM-9 (ATCC CCL-159) and RPMI 8226 (ATCC TBI-196) were purchased from the American Type Culture Collection (ATCC, Rockville, MD, USA). Cells were cultured in RPMI 1640 medium (Gibco Laboratories) supplemented with 10% heat-inactivated fetal bovine serum (Gibco Laboratories) and 1% penicillin/streptomycin (Thermo Fisher Scientific). Cells were cultured in a constant-temperature incubator at 37° C. under 5% CO₂ and subcultured at intervals of 3 to 4 days.

### Cell Viability Assay

KBB-N2 with a purity of 98% or higher was purchased from Chengdu Biopurity Phytochemicals Ltd. Korea (Seoul, Korea). KBB-N2 was dissolved in dimethyl sulfoxide (DMSO) to make a 100 mM stock, which was then stored at −20° C. and diluted in medium before use. Multiple myeloma cell lines U266, IM-9, and RPMI 8226 were each treated with KBB-N2 at the same concentration of 50 μM, suspended in 100 μL of culture medium, seeded into a 96-well plate (5×10⁵ cells/well), and then cultured for 24 hours. Thereafter, 10 μL of EZ-CyTox was mixed with the culture medium using a cell viability assay kit (EZ-CyTox, Daeil Lab Service Co., Seoul, Korea) and incubated in a dark room at 37° C. under 5% CO₂ for 1 hour.

As EZ-CYTOX reacts with cellular dehydrogenase to produce orange water-soluble formazan, the absorbance was measured at 450 nm using a VERSA Max Micro plate reader (Molecular Devices, Sunnyvale, CA, USA). The formula for calculating the percentage of viable cells in ginsenoside-treated cells relative to untreated cells is as follows.

$$Cell viability ( \% ) = [ OD ( KBB - N 2 - treated cells ) - OD ( blank ) ] \text{} / [ OD ( cells ) - OD ( blank ) ] \times 100$$

### Cell Death Assay

Multiple myeloma cells IM-9 and RPMI 8226 (3×10⁶ cells/mL) treated with KBB-N2 for 48 hours were washed with 1x PBS (phosphate-buffered saline, pH 7.4) and fixed with 70% ethanol. After washing several times with 1×PBS, 5 μL of Annexin V-FITC and 5 μL of PI (propidium iodine) (Sigma-Aldrich, St. Louis, MO) were added to 500 μL of 1×PBS containing the cells (1×10⁶ cells/mL) suspended therein, followed by staining in a dark room at room temperature for 30 minutes. Then, 500 μL of 1× binding buffer was added thereto, and flow cytometry was performed using FACSCalibur (Becton Dickinson, Franklin Lakes, NJ, USA), followed by analysis using the CellQuest software (Becton Dickinson) program.

### Evaluation of Effectiveness of KBB-N2 on Treatment of Multiple Myeloma Using Multiple Myeloma Mouse Model

Using xenograft models obtained by injecting the same human-derived multiple myeloma cell lines ARH77, U266, and RPMI 8226 into mice, each of KBB-N2 was injected into the vein tail at 7 mg/Kg/day, and as a positive control, lenalidomide, which is currently widely used as a therapeutic agent for multiple myeloma in clinical practice, was injected into the vein tail at 1 mg/Kg/day. As a result, it was found that KBB-N2 had a therapeutic effect equal to or higher than that of traditional lenalidomide for multiple myeloma. In addition, the effect of KBB-N2 on multiple myeloma treatment was also found in a bone marrow biopsy after treatment (FIG. 9).

### Confirmation of Non-Toxicity of KBB-N2 to Normal Hematopoietic Stem Cells

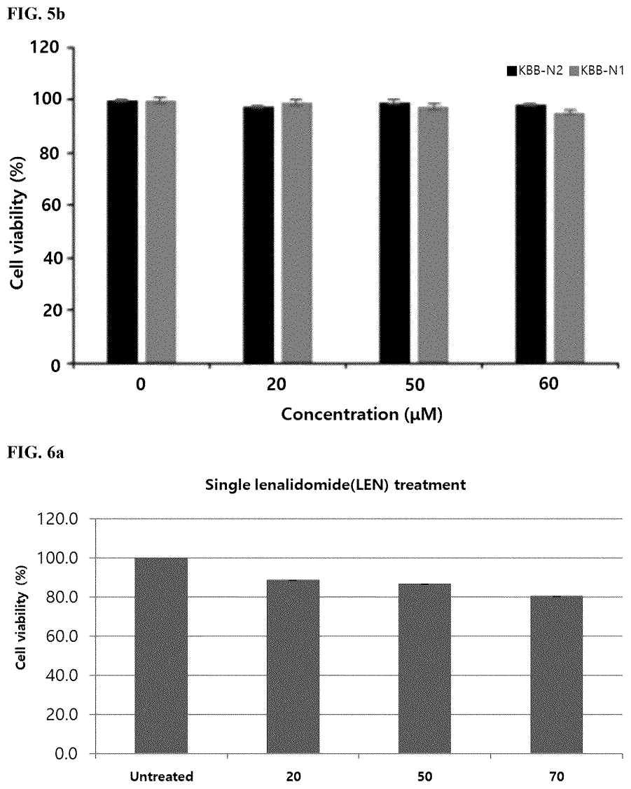

G-CSF was subcutaneously injected into a normal donor to mobilize hematopoietic stem cells, and a peripheral blood stem cell (PBSC) fraction was extracted from the donor's peripheral blood by leukapheresis. The obtained normal PBSC fraction and KBB-N2 were cultured on methylcellulose for 2 weeks, and the cytotoxicity of KBB-N2 to hematopoietic stem cells was evaluated by counting the number of the formed erythroid progenitor colonies and leukocyte/megakaryocytic progenitor colonies. As a result, it was confirmed that, even when the concentration of KBB-N2 used was increased up to 60 μM, the number and morphology/formation of the progenitor cell colonies were not significantly inhibited or abnormal findings were not observed, indicating that the compound of the present invention is not toxic to normal hematopoietic stem cells (FIG. 5).

Co-Treatment with KBB-N2 and Lenalidomide

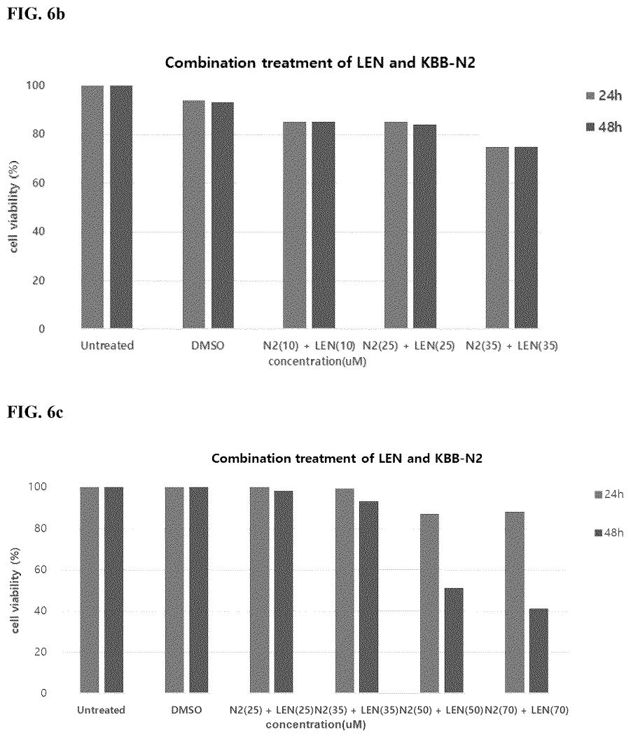

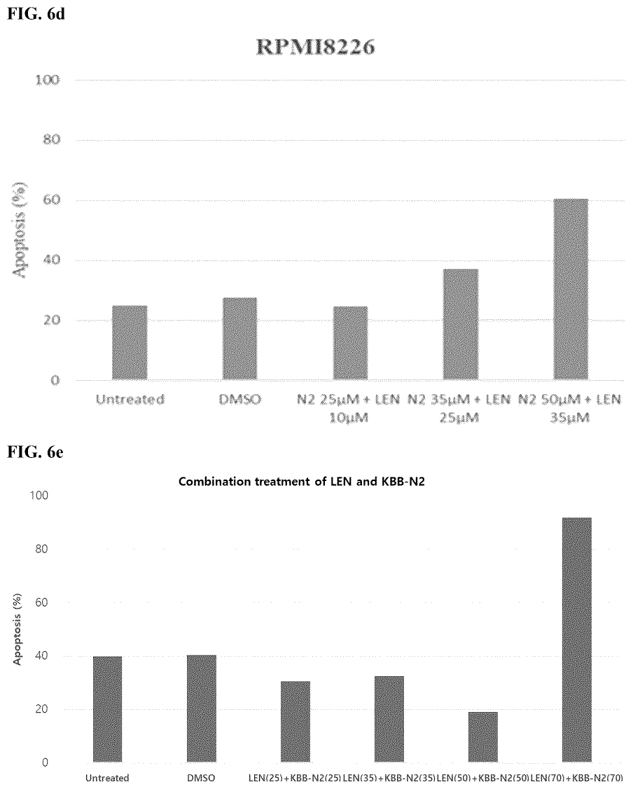

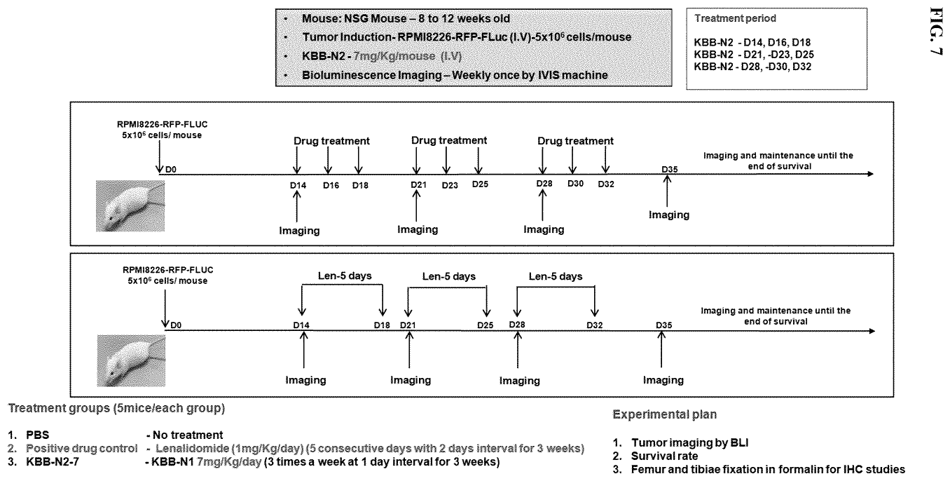

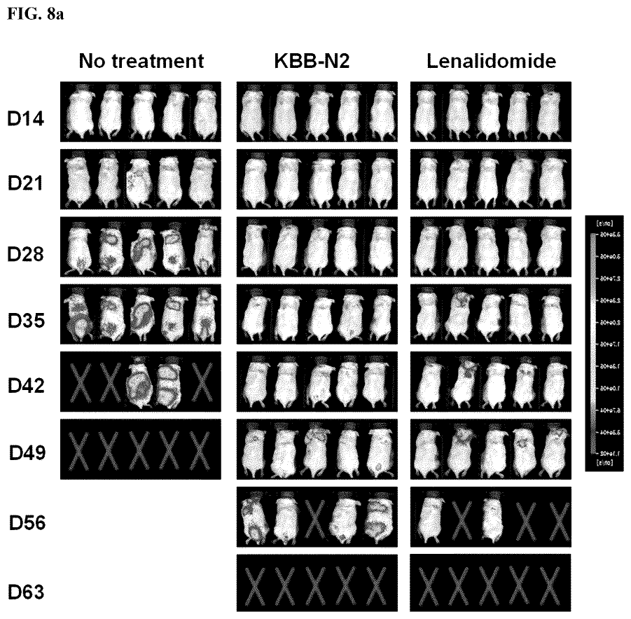

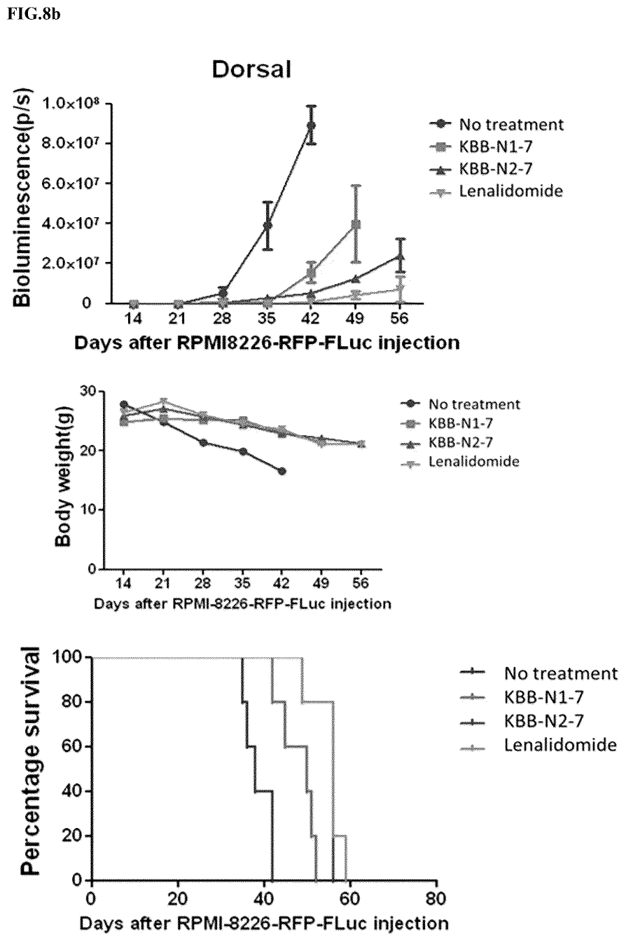

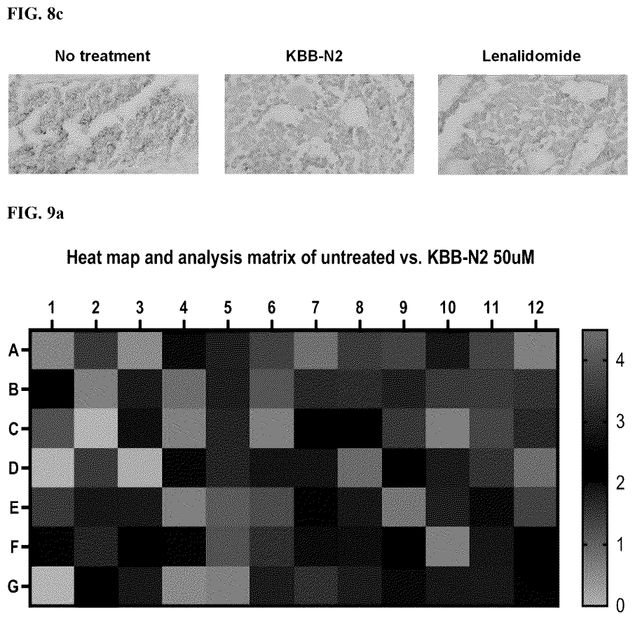

It was confirmed that the killing effect was higher when multiple myeloma cell lines were co-treated with lenalidomide and KBB-N2 than when treated with lenalidomide alone (FIG. 6).

### Investigation of KBB-N2-Induced Death Mechanism of Multiple Myeloma Cell Line

To investigate the cell death mechanism induced by treatment with KBB-N2, changes in the expression of apoptosis-related genes in a multiple myeloma cell line were measured. 50 μM of KBB-N2 was added to multiple myeloma cells (million cells/mL), followed by 48 hours of culture. Then, total RNA was extracted and the expression levels of 84 apoptosis-related genes were analyzed using quantitative RT-PCR array (Cat #PAHS-012Z; Qiagen Seoul, Seoul, Korea). After treating the cells with 50 μM of KBB-N2, the cells were cultured for 48 hours, and total RNA was isolated from the RPMI 8226 cells using the RNAqueous total RNA isolation kit (Thermo Fisher Scientific). Contaminating genomic DNA in the extracted RNA was removed using an RNase-Free DNase set (Qiagen). Total RNA (5 ug) was reverse transcribed into cDNA in a reaction volume of 10 μL using the RT2 First Strand Kit (Qiagen). Next, 10 μL of cDNA was combined with each primer set of the human apoptosis-related RT2 Profiler PCR Array (Qiagen) according to the manufacturer's protocol. Amplification reactions were performed using the real-time polymerase chain reaction system CFX96 (Bio-Rad, Hercules, CA, USA) with automated baseline and threshold cycle detection. The relative expression of growth factor-related genes was normalized to five built-in housekeeping genes. Each group was analyzed by the web-based software GeneGlobe Data Analysis Center (Qiagen).

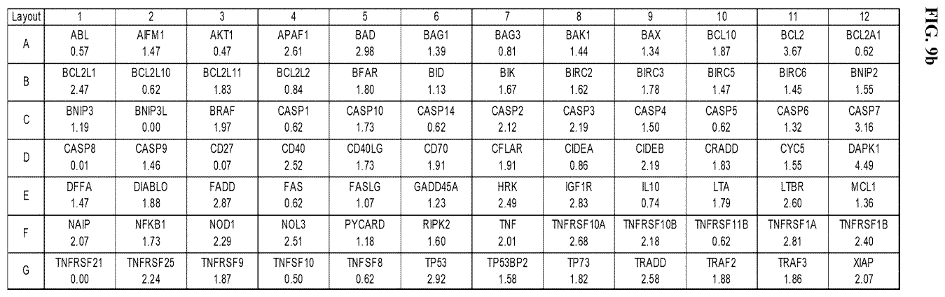

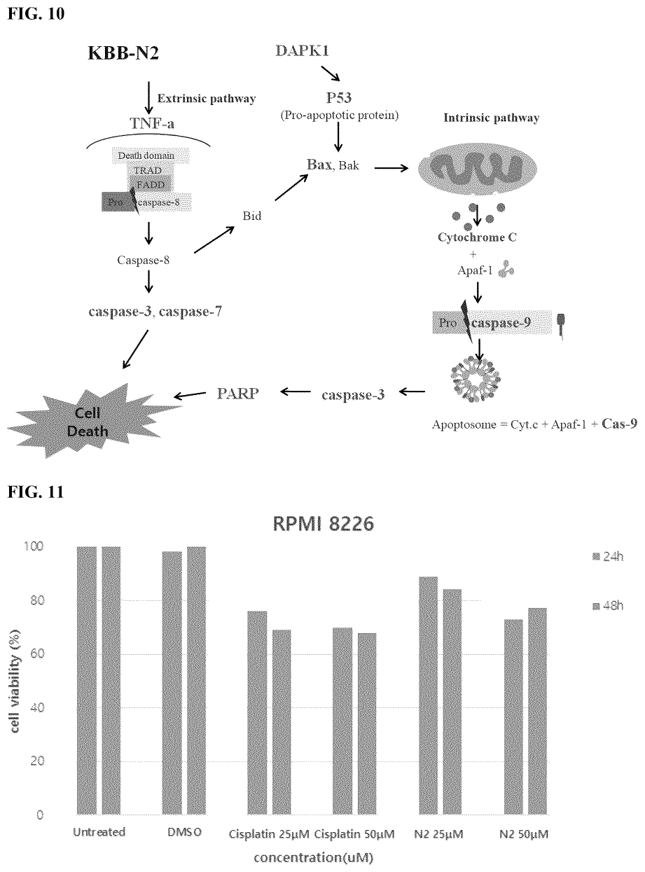

As a result of the analysis performed by performing the PCR array after adding 50 μM of KBB-N2 to the multiple myeloma cell line, it was confirmed that the expression of the following apoptosis-related genes was significantly changed (FIG. 10):

Apoptosis-related genes overexpressed by at least 4-fold: DAPK1

Apoptosis-related genes overexpressed by at least 3-fold: BCL2, and CAS7

Apoptosis-related genes overexpressed by at least 2-fold: APAF1, BAD, BCL2L1, CASP2, CASP3, FADD, HRK, IGFIR, LTBR, NAIP, NOD1, NOL3, TNF, TNFRSF10A, TNFRSF10B, TNFRF1A, TNFRSF1B, TNFRSF25, TP53, TRADD, and XIAP

Although the present invention has been described in detail with reference to the specific features, it will be apparent to those skilled in the art that this description is only of a preferred embodiment thereof, and does not limit the scope of the present invention. Thus, the substantial scope of the present invention will be defined by the appended claims and equivalents thereto.

1. A method for preventing or treating cancer, wherein the method comprises:
administering a composition comprising a compound represented by the following Formula 1 or a pharmaceutically acceptable salt thereof as an active ingredient to a subject in need thereof: 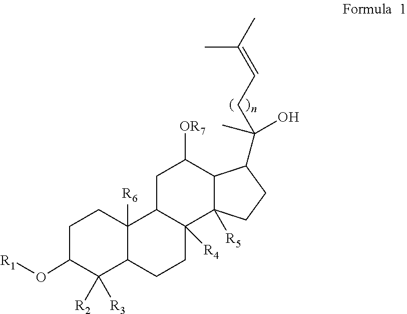
wherein R₁ is 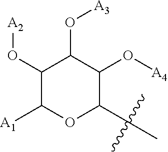
(where A₁ is C₁-C₃ alkyl unsubstituted or substituted with hydroxy, and A₂ to A₄ are each independently hydrogen or C₁-C₃ alkyl); R₂ to R₇ are each independently hydrogen or C₁-C₃ alkyl; and n is an integer ranging from 1 to 3.

2. The method according to claim 1, wherein A₁ is hydroxymethyl, and A₂ to A₄ are hydrogen.

3. The method according to claim 1, wherein R₂ to R₆ are each independently hydrogen or methyl.

4. The method according to claim 1, wherein n is 2.

5. The method according to claim 1, wherein the compound represented by Formula 1 is a compound represented by the following Formula 2:

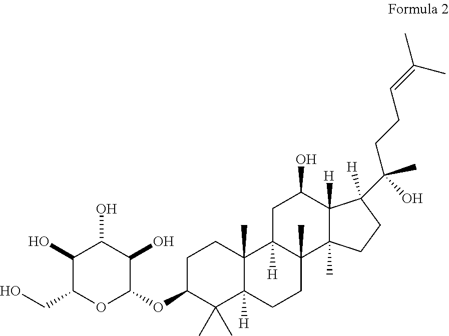

6. The method according to claim 1, wherein the cancer is multiple myeloma.

7. The method according to claim 1, further comprising administering a compound represented by the following Formula 3 or a pharmaceutically acceptable salt thereof to the subject: 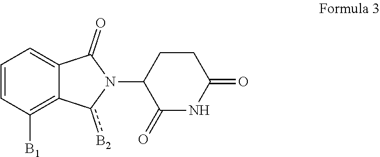
wherein B₁ is hydrogen or —NH₂, [symbol] represents a single bond or double bond, and B₂ is hydrogen when [symbol] represents a single bond, or oxygen when [symbol] represents a double bond.

8. A method for preventing or treating cancer, wherein the method comprises:
co-administering a compound represented by the following Formula 3 or a pharmaceutically acceptable salt thereof, with a compound represented by the following Formula 1 or a pharmaceutically acceptable salt thereof to a subject in need thereof: 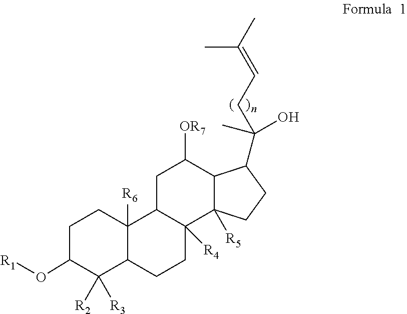
wherein R₁ is 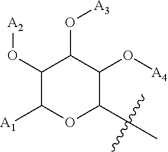
(where A₁ is C₁-C₃ alkyl unsubstituted or substituted with hydroxy, and A₂ to A₄ are each independently hydrogen or C₁-C₃ alkyl); R₂ to R₇ are each independently hydrogen or C₁-C₃ alkyl; and n is an integer ranging from 1 to 3; 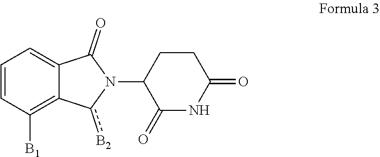
wherein B₁ is hydrogen or —NH₂, [symbol] represents a single bond or double bond, and B₂ is hydrogen when [symbol] represents a single bond, or oxygen when [symbol] represents a double bond.

9. The method according to claim 8, wherein the cancer is multiple myeloma.
